(19) United States
(12) Patent Application Publication
(10) Pub. No.: US 2022/0362486 A1
(43) Pub. Date: Nov. 17, 2022

(54) Safe Syringe Device

(71) Applicant: Kelly Wonser, Goessel, KS (US)

(72) Inventor: Kelly Wonser, Goessel, KS (US)

(21) Appl. No.: 17/741,048

(22) Filed: May 10, 2022

Related U.S. Application Data
(60) Provisional application No. 63/188,102, filed on May 13, 2021.

(51) Int. Cl. A61M 5/32

(52) U.S. Cl. CPC A61M 5/3278; A61M 2005/3125

(57) ABSTRACT

The present invention relates to a novel safety syringe device. The device is a medication distribution system equipped with a bendable needle. The device comprises a syringe featuring a button-activated, bendable needle that can bend inwards toward the syringe after use. Further, the bent needle fits flush against the side of the syringe, inside a small, guided chamber. This allows a user to safely re-cap and discard a needle while eliminating the possibility of puncturing themselves with the needle.

### CROSS-REFERENCE TO RELATED APPLICATION

The present application claims priority to, and the benefit of, U.S. Provisional Application No. 63/188,102, which was filed on May 13, 2021, and is incorporated herein by reference in its entirety.

### FIELD OF THE INVENTION

The present invention relates generally to safety syringe devices. More specifically, the present invention relates to an improved safety syringe device that provides users with a bendable needle used for safe disposal. The device includes a chamber to house the needle once bent to ensure the pointed end cannot cause injury. Accordingly, the present disclosure makes specific reference thereto. Nonetheless, it is to be appreciated that aspects of the present invention are also equally applicable to other like applications, devices and methods of manufacture.

### BACKGROUND

By way of background, this invention relates to improvements in syringes. Current needles and syringes can be dangerous to throw away. Once a needle is applied and medication is distributed, users may be at risk for additional needle pricks and other health hazards that can injure a user or spread illness. Further, applying safety caps can be difficult and inefficient when dealing with patients in given timeframes. Thus, a safer syringe is necessary for all users who utilize syringes.

Furthermore, it is typical for many syringes to be supplied prefilled to end users for self-injection, for instance for diabetics. The use of the syringe in untrained hands, particularly with older patients, may lead to damage to the needle before or after use, as well as additional needle pricks. Furthermore, even if the protective cap can be removed, the unskilled user often cannot replace the protective cap. Accordingly, a safer syringe to prevent injuries is necessary.

Thus, it is necessary for users that utilize syringes to be able to discard the needle while preventing injuries and illness from spreading. Generally, the use of syringes can create a risk of additional needle pricks and other health hazards. Therefore, users require a way to ensure the pointed end cannot cause injury prior to disposal in a trash receptacle.

Therefore, there exists a long-felt need in the art for a safety syringe device that provides users with an improved syringe and needle system with a bendable needle used for safe disposal. There is also a long-felt need in the art for a safety syringe device that allows the needle to be applied, medication distributed, and then bent flush against the syringe via a push button. Further, there is a long-felt need in the art for a safety syringe device that includes a chamber to house the needle once bent against the syringe. Moreover, there is a long-felt need in the art for a device that ensures the pointed end cannot cause injury prior to disposal in a trash receptacle. Further, there is a long-felt need in the art for a safety syringe device that enables users to safely recap and discard the needle while preventing injuries and illness from spreading. Finally, there is a long-felt need in the art for a safety syringe device that allows users to easily inject medication without worry of accidentally hurting themselves prior to disposal.

The subject matter disclosed and claimed herein, in one embodiment thereof, comprises a safety syringe device. The device is a medication distribution system equipped with a bendable needle. The device comprises a syringe featuring a button-activated, bendable needle that can bend inwards toward the syringe after use. Further, the bent needle fits flush against the side of the syringe, inside a small, guided chamber. This allows a user to safely re-cap and discard a needle or insulin needle while eliminating the possibility of puncturing themselves with the needle, while attempting to discard it. Users can easily inject medication, insulin, etc., without worry of accidentally hurting themselves prior to disposal.

In this manner, the safety syringe device of the present invention accomplishes all of the foregoing objectives and provides users with a device that prevents injuries while discarding a needle. The device allows a user to apply a needle, distribute medication, and then bend the needle flush against a syringe via a push button. The device saves time and effort and prevents injuries and illness from spreading.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a safety syringe device. The device is a medication distribution system equipped with a bendable needle. The device comprises a syringe featuring a button-activated, bendable needle that can bend inwards toward the syringe after use. Further, the bent needle fits flush against the side of the syringe, inside a small, guided chamber. This allows a user to safely re-cap and discard a needle while eliminating the possibility of puncturing themselves with the needle. Users can easily inject medication, insulin, etc., without worry of accidentally hurting themselves prior to disposal.

In one embodiment, the syringe comprises a cylindrical housing with an internal cavity and conduit connecting into the bendable needle. Further, the syringe comprises a plunger means for introducing irrigating or medicating fluid through the conduit into the bendable needle.

In one embodiment, the bendable needle is a flexible, hollow needle that extends between a proximal connection end and a distal fluid discharge end. The flexible, syringe needle comprises a plunger means for introducing irrigating or medicating fluid through the conduit into the hollow needle. The plunger means is removably connected to the proximal connection end of the flexible needle, such that the device can be reused. Further, the flexible needle comprises an open tip end which is tapered.

In another embodiment, a flexible syringe needle may have the needle and plunger means integrally connected so that the entire syringe is disposable after use.

In another embodiment, the syringe comprises at least one measuring marking on an exterior surface thereof to visually verify a working length thereof, or on the housing.

In one embodiment, the bendable needle bends inwards toward the syringe housing after use such that the needle sits flush against the housing of the syringe. In one embodiment, a button positioned on the syringe housing slides forward to bend the needle toward the body of the syringe. Specifically, a user slides the button upwards toward the needle, wherein the button engages a slider component that contacts the needle, bending it. The slider component guides the needle toward the side of the syringe housing, wherein in one embodiment, the needle contacts a chamber. The chamber is positioned on the side of the syringe housing. The chamber is sized to accept the needle and comprises a securing means for retaining the needle. The securing means can be any suitable securing means as is known in the art, such as a snap lock or ratcheting lock, etc.

In one embodiment, the flexible needle may comprise a plurality of user selectable needles (not shown) having a range of aperture sizes for user determination of fluid dispensing flow rate according to user selected fluid dispensing aperture size.

In one embodiment, the flexible needle is made of plastics, such as polyethylene or polypropylene. The needle may also be manufactured of a silicone, or a flexible metal, such as nitinol.

In yet another embodiment, the flexible syringe needle may include a chain-like plurality (not shown) of articulated rigid segments having a fluid delivery tube running longitudinally there through, wherein the segments and tube are comprised of a pharmaceutically suitable material.

In another embodiment, the safety syringe device comprises a plurality of indicia on the syringe housing and/or needle.

In yet another embodiment, a method of utilizing a safety syringe device while preventing injuries prior to disposal in a trash receptacle is disclosed. The method includes the steps of providing a safety syringe device comprising a button-activated, bendable needle that bends inward and fits flush against the syringe inside a guided chamber after use. The method also comprises applying the bendable needle of the device to a user, such that the user is injected with the needle. Further, the method comprises distributing medicine via the needle to the user, wherein the plunger of the syringe is depressed to distribute the medicine. The method also comprises bending the needle flush against the syringe for disposal and securing the needle in the chamber. Finally, safely disposing of the safety syringe device without injury.

In yet another embodiment of the present invention, a safety syringe device is disclosed for securing a needle of a syringe before disposing it. Specifically, the device is a medication distribution system equipped with a bendable needle. A user would utilize the syringe in the conventional method, and then bend the needle to the syringe housing. Typically, the user would slide a button positioned on the syringe housing upwards toward the needle. The button would engage the slider component which would contact the needle. The slider component then bends the needle toward the syringe housing, guiding the needle into the chamber. The chamber retains the needle, protecting the user from an unintended needle stick. This allows a user to safely re-cap and discard a needle, while eliminating the possibility of puncturing themselves with the needle. Thus, users can easily inject medication, insulin, etc., without worry of accidentally hurting themselves prior to disposal.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of the safety syringe device of the present invention in accordance with the disclosed architecture;

FIG. 2 illustrates a perspective view of one potential embodiment of the safety syringe device of the present invention, wherein the sliding button is activated to bend the needle flush against the syringe in accordance with the disclosed architecture;

FIG. 3 illustrates a perspective view of one potential embodiment of the safety syringe device of the present invention, wherein the needle is safely stored in a small, guided chamber in accordance with the disclosed architecture;

FIG. 4 illustrates a perspective view of one potential embodiment of the chamber of the safety syringe device of the present invention in accordance with the disclosed architecture; and

FIG. 5 illustrates a flowchart of one potential method of utilizing the safety syringe device of the present invention while preventing injuries prior to disposal in a trash receptacle in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a safety syringe device that provides users with an improved syringe and needle system with a bendable needle used for safe disposal. There is also a long-felt need in the art for a safety syringe device that allows the needle to be applied, medication distributed, and then bent flush against the syringe via a push button. Further, there is a long-felt need in the art for a safety syringe device that includes a chamber to house the needle once bent against the syringe. Moreover, there is a long-felt need in the art for a device that ensures the pointed end cannot cause injury prior to disposal in a trash receptacle. Further, there is a long-felt need in the art for a safety syringe device that enables users to safely recap and discard the needle while preventing injuries and illness from spreading. Finally, there is a long-felt need in the art for a safety syringe device that allows users to easily inject medication without worry of accidentally hurting themselves prior to disposal.

The present invention, in one exemplary embodiment, is a novel safety syringe device. The device is a medication distribution system equipped with a bendable needle. The device comprises a syringe featuring a button-activated, bendable needle that can bend inwards toward the syringe after use. Further, the bent needle fits flush against the side of the syringe inside a small, guided chamber. This allows a user to safely re-cap and discard a needle while eliminating the possibility of puncturing themselves with the needle. The present invention also includes a novel method of utilizing a safety syringe device while preventing injuries prior to disposal in a trash receptacle. The method includes the steps of providing a safety syringe device comprising a button-activated, bendable needle that bends inward and fits flush against the syringe inside a guided chamber after use. The method also comprises applying the bendable needle of the device to a user, such that the user is injected with the needle. Further, the method comprises distributing medicine via the needle to the user, wherein the plunger of the syringe is depressed to distribute the medicine. The method also comprises bending the needle flush against the syringe for disposal and securing the needle in the chamber. Finally, safely disposing of the safety syringe device without injury.

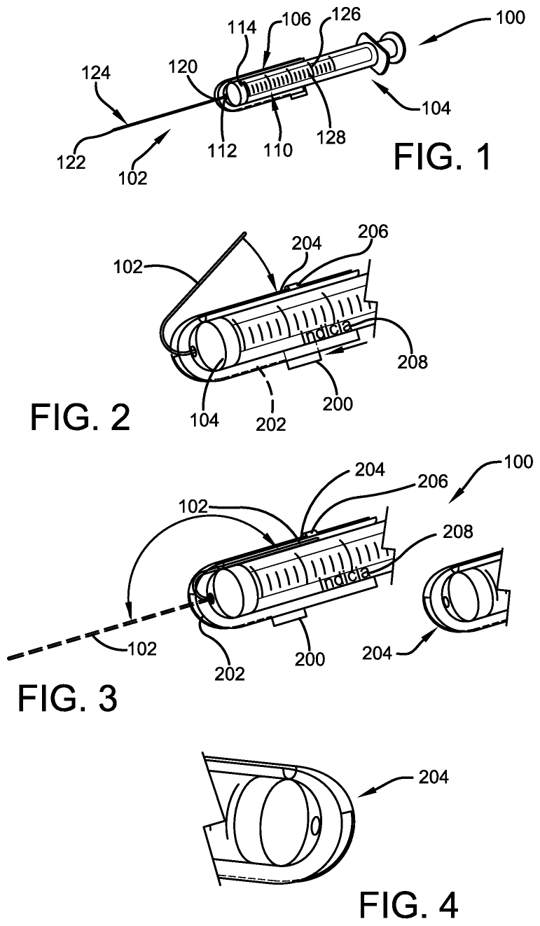

Referring initially to the drawings, FIG. 1 illustrates a perspective view of one embodiment of the safety syringe device 100 of the present invention. In the present embodiment, the safety syringe device 100 is an improved safety syringe device 100 that enables users to safely recap and discard a needle without injury. The device 100 is especially designed to allow patients, nurses, etc., or any other suitable user as is known in the art, to easily discard needles while preventing injuries and illness from spreading. More specifically, the device 100 comprises a button-activated, bendable needle 102 that can be bent inwards toward the syringe 104 after use. The bent needle 102 can fit flush against the side of the syringe 104, inside a small, guided chamber 106.

In one embodiment, the syringe 104 comprises a cylindrical housing 108 with an internal cavity 110 and conduit 112 connecting into the bendable needle 102. The housing 108 can be any suitable shape as is known in the art, such as cylindrical, square, rectangular, etc., depending on the needs and/or wants of a user. The internal cavity 110 retains the irrigation fluid and/or medication for dispensing. Once filled, the fluid is dispensed through the conduit 112 into the bendable needle 102.

Additionally, the syringe 104 comprises a plunger means 114 within the internal cavity 110. The plunger means 114 comprises a flexible, rubber-sealing member 116 for introducing the irrigating or medicating fluid through the conduit 112 into the bendable needle 102. The plunger means 114 can be placed into the internal cavity 110 of the syringe housing 108 via a rear opening 118, which has a larger cross-sectional area. The outside diameter of the flexible, rubber-sealing member 116 is slightly larger than the inside diameter of the internal cavity 110, such that the medical fluid in the syringe 104 can flow out from the bendable needle 102, while the plunger means 114 is pushed from the back end thereof. Similarly, medical fluids can be drawn into the syringe 104 by pulling the plunger means 114 backwards.

Furthermore, the bendable needle 102 is a flexible, hollow needle 102 that extends between a proximal connection end 120 and a distal fluid discharge end 122. The flexible, syringe needle 102 utilizes the plunger means 114 for introducing irrigating or medicating fluid through the conduit 112 into the hollow needle 102. The plunger means 114 is removably connected to the proximal connection end 120 of the flexible needle 102, such that the device 100 can be reused. The plunger means 114 can be removably secured via any suitable securing means as is known in the art, such as screwing, snap fit, etc. Further, the flexible needle 102 comprises an open tip end 124 which is tapered.

In another embodiment, a flexible syringe needle 102 may have the needle 102 and plunger means 114 integrally connected, so that the entire syringe 104 is disposable after use.

In another embodiment, the syringe 104 comprises at least one measuring marking 126 on an exterior surface thereof to visually verify a working length thereof. The at least one measuring marking 126 on an exterior surface thereof comprises a plurality of additional markings 128 thereon, such as tick marks, dashes, lines, numbers, etc., or any other suitable markings as is known in the art.

FIGS. 2-4 illustrate a perspective view of another embodiment of the safety syringe device 100 of the present invention. In this embodiment, the bendable needle 102 bends inwards toward the syringe housing 108 after use, such that the needle 102 sits flush against the housing 108 of the syringe 104. Further, a button 200 positioned on the syringe housing 108 slides forward to bend the needle 102 toward the body 108 of the syringe 104. Specifically, a user slides the button 200 upwards toward the needle 102, wherein the button 200 engages a slider component 202 that contacts the needle 102, bending it. The slider component 202 guides the needle 102 toward the side of the syringe housing 108, wherein in one embodiment, the needle 102 contacts a chamber 204. The chamber 204 is positioned on the side of the syringe housing 108. The chamber 204 is sized to accept the needle 102 and comprises a securing means 206 for retaining the needle 102. The securing means 206 can be any suitable securing means as is known in the art, such as a snap lock or ratcheting lock, etc., depending on the needs and/or wants of a user. Additionally, the user can manually bend the needle 102 and secure it within the chamber 204 for disposal, without using the button 200 and slider component 202.

In one embodiment, the flexible needle 102 may comprise a plurality of user selectable needles (not shown) having a range of aperture sizes for user determination of fluid dispensing flow rate according to user selected fluid dispensing aperture size.

In another embodiment, the flexible needle 102 is made of plastics, such as polyethylene or polypropylene. The needle 102 may also be manufactured of a silicone, or a flexible metal, such as nitinol, etc., or any other suitable materials as is known in the art.

In yet another embodiment, the flexible syringe needle 102 may include a chain-like plurality (not shown) of articulated rigid segments having a fluid delivery tube running longitudinally therethrough, wherein the segments and tube are comprised of a pharmaceutically suitable material. This allows the user to manually bend the needle 102 and secure it within the chamber 204 for disposal without using the button 200 and slider component 202.

In another embodiment, the safety syringe device 100 comprises a plurality of indicia 208 on the syringe housing 108 and/or needle 102. For example, the syringe 104 of the device 100 may include advertising, a trademark, or other letters, designs, or characters, printed, painted, stamped, or integrated into the syringe 104, or any other indicia 208 as is known in the art. Specifically, any suitable indicia 208 as is known in the art can be included, such as but not limited to, patterns, logos, emblems, images, symbols, designs, letters, words, characters, animals, advertisements, brands, etc., that may or may not be medical or brand related.

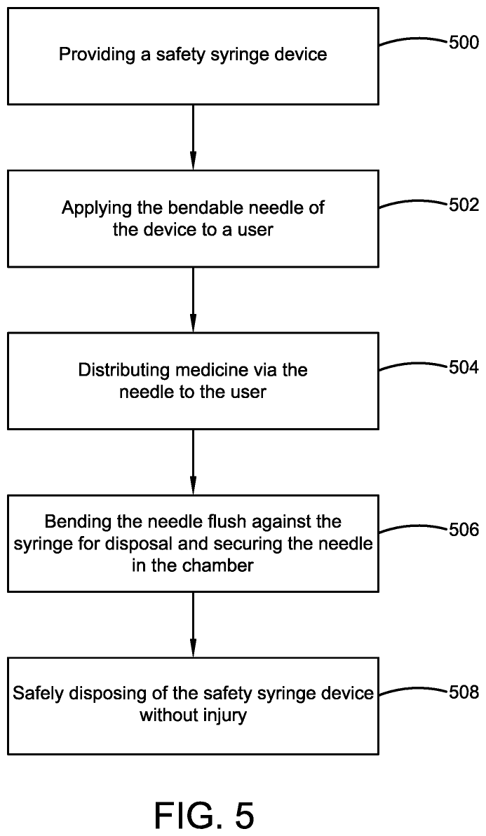

FIG. 5 illustrates a flowchart of the method of utilizing a safety syringe device while preventing injuries prior to disposal in a trash receptacle. The method includes the steps of at 500, providing a safety syringe device comprising a button-activated, bendable needle that bends inward and fits flush against the syringe, inside a guided chamber after use. The method also comprises at 502, applying the bendable needle of the device to a user, such that the user is injected with the needle. Further, the method comprises at 504, distributing medicine via the needle to the user, wherein the plunger of the syringe is depressed to distribute the medicine. The method also comprises at 506, bending the needle flush against the syringe for disposal and securing the needle in the chamber. Finally, at 508, safely disposing of the safety syringe device without injury.

In yet another embodiment of the present invention, a safety syringe device 100 is disclosed for securing a needle 102 of a syringe 104 before disposing it. Specifically, the device 100 is a medication distribution system equipped with a bendable needle 102. A user would utilize the syringe 104 in the conventional method, and then bend the needle 102 to the syringe housing 108. Typically, the user would slide a button 200 positioned on the syringe housing 108 upwards toward the needle 102. The button 200 would engage the slider component 202 which would contact the needle 102. The slider component 202 then bends the needle 102 toward the syringe housing 108 guiding the needle 102 into the chamber 204. The chamber 204 retains the needle 102 protecting the user from an unintended needle stick. This allows a user to safely re-cap and discard a needle 102 while eliminating the possibility of puncturing themselves with the needle 102. Thus, users can easily inject medication, insulin, etc., without worry of accidentally hurting themselves prior to disposal.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “safety syringe device” and “device” are interchangeable and refer to the safety syringe device 100 of the present invention.

Notwithstanding the foregoing, the safety syringe device 100 of the present invention can be of any suitable size and configuration as is known in the art without affecting the overall concept of the invention, provided that it accomplishes the above-stated objectives. One of ordinary skill in the art will appreciate that the safety syringe device 100 as shown in FIGS. 1-5 is for illustrative purposes only, and that many other sizes and shapes of the safety syringe device 100 are well within the scope of the present disclosure. Although the dimensions of the safety syringe device 100 are important design parameters for user convenience, the safety syringe device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A safety syringe device that enables a user to safely recap and discard a needle without incurring an injury, the safety syringe device comprising:
a syringe having a bendable needle;
wherein the syringe comprises a housing with an internal cavity and a conduit connecting into the bendable needle; and
further wherein the bendable needle is bent inwards toward the syringe after use.

2. The safety syringe device of claim 1, wherein the housing comprises a plunger means within the internal cavity.

3. The safety syringe device of claim 2, wherein the plunger means comprises a rubber-sealing member.

4. The safety syringe device of claim 3, wherein the bendable needle is a hollow needle.

5. The safety syringe device of claim 4, wherein the bendable needle extends between a proximal connection end and a distal fluid discharge end.

6. The safety syringe device of claim 5, wherein the plunger means introduces a fluid through the conduit and into the bendable needle.

7. The safety syringe device of claim 6, wherein the housing comprises at least one measuring marking.

8. The safety syringe device of claim 1, wherein the bendable needle fits flush against a side of the syringe.

9. The safety syringe device of claim 8, wherein the bendable needle fits inside a chamber on the syringe.

10. The safety syringe device of claim 9, wherein the bendable needle is bent via a button positioned on the syringe.

11. The safety syringe device of claim 10, wherein the button slides forward to bend the bendable needle.

12. The safety syringe device of claim 11, wherein the button engages a slider component that contacts the bendable needle to bend the same.

13. The safety syringe device of claim 12, wherein the slider component guides the bendable needle toward the housing and into the chamber.

14. A safety syringe device that enables a user to safely recap and discard a needle without incurring an injury, the safety syringe device comprising:
a syringe with a bendable needle;
wherein the syringe comprises a housing with an internal cavity and conduit connecting into the bendable needle;
wherein the housing comprises a plunger means with a rubber-sealing member within the internal cavity;
wherein the bendable needle is bent inwards toward the syringe after use;
wherein a button slides forward to bend the bendable needle;
wherein the button engages a slider component that contacts the bendable needle, bending it; and
further wherein the slider component guides the bendable needle toward the housing and into the chamber.

15. The safety syringe device of claim 14 further comprising a plurality of indicia.

16. The safety syringe device of claim 14, wherein the housing comprises at least one measuring marking.

17. The safety syringe device of claim 14, wherein the bendable needle fits flush against a side of the syringe.

18. The safety syringe device of claim 14, wherein the bendable needle is a hollow needle.

19. The safety syringe device of claim 14, wherein the bendable needle extends between a proximal connection end and a distal fluid discharge end.

20. A method of utilizing a safety syringe device to prevent an injury prior to disposal in a trash receptacle, the comprising the steps of:
providing a safety syringe device comprising a button-activated, bendable needle that bends inward and fits flush against the syringe, inside a guided chamber after use;
applying the bendable needle of the device to a user, such that the user is injected with the needle;
distributing a medicine via the needle to the user, wherein the plunger of the syringe is depressed to distribute the medicine;
bending the needle flush against the syringe for disposal and securing the needle in the chamber; and
safely disposing of the safety syringe device without injury.
